(19) United States
(12) Patent Application Publication
(10) Pub. No.: US 2006/0190011 A1
(43) Pub. Date: Aug. 24, 2006

(54) Systems and methods for providing a reference plane for mounting an acetabular cup during a computer-aided surgery

(76) Inventor: Michael Ries, Tiburon, CA (US)

Correspondence Address: CHIEF PATENT COUNSEL;SMITH & NEPHEW, INC., 1450 BROOKS ROAD, MEMPHIS, TN, 38116, US

(21) Appl. No.: 11/292,710

(22) Filed: Dec. 1, 2005

Related U.S. Application Data
(60) Provisional application No. 60/632,627, filed on Dec. 2, 2004.

(51) Int. Cl. A61B 19/00; A61B 17/00

(52) U.S. Cl. 606/130; 606/1

(57) ABSTRACT

Systems and methods for providing a reference for an acetabular cup plane in accordance with embodiments of the invention can include a method performed by a computer-aided surgical navigational system with a display screen and at least one sensor. The method can include detecting at least one array associated with a platform upon which the patient is supported in a supine position. The method can also include defining a surgical reference plane for a surgical procedure associated with an acetabular component, based at least in part on detecting the array associated with the upper surface of the platform. Furthermore, the method can include outputting via the screen at least one user interface for use with the surgical procedure associated with an acetabular component, based at least in part on detecting the array associated with the upper surface of the platform.

### RELATED APPLICATIONS

This application claims priority to U.S. Provisional Ser. No. 60/632,627, entitled “Table Reference for Cup Plane,” filed on Dec. 2, 2004, which is incorporated by reference.

### FIELD OF THE INVENTION

The invention relates generally to systems and methods related to computer aided-surgery, and more specifically to systems and methods for providing a reference plane for mounting an acetabular cup plane during a computer-aided surgery.

### BACKGROUND OF THE INVENTION

Many surgical procedures require a wide array of instrumentation and other surgical items. Such items may include, but are not limited to: sleeves to serve as entry tools, working channels, drill guides and tissue protectors; scalpels; entry awls; guide pins; reamers; reducers; distractors; guide rods; endoscopes; arthroscopes; saws; drills; screwdrivers; awls; taps; osteotomes, wrenches, trial implants and cutting guides. In many surgical procedures, including orthopedic procedures, it may be desirable to associate some or all of these items with a guide and/or handle incorporating a navigational reference, allowing the instrument to be used with a computer-aided surgical navigation system.

Several manufacturers currently produce computer-aided surgical navigation systems. The TREON™ and ION™ systems with FLUORONAV™ software manufactured by Medtronic Surgical Navigation Technologies, Inc. are examples of such systems. The BrainLAB VECTORVISION™ system is another example of such a surgical navigation system. Systems and processes for accomplishing computer-aided surgery are also disclosed in U.S. Ser. No. 10/084,012, filed Feb. 27, 2002 and entitled “Total Knee Arthroplasty Systems and Processes”; U.S. Ser. No. 10/084,278, filed Feb. 27, 2002 and entitled “Surgical Navigation Systems and Processes for Unicompartmental Knee Arthroplasty”; U.S. Ser. No. 10/084,291, filed Feb. 27, 2002 and entitled “Surgical Navigation Systems and Processes for High Tibial Osteotomy”; International Application No. US02/05955, filed Feb. 27, 2002 and entitled “Total Knee Arthroplasty Systems and Processes”; International Application No. US02/05956, filed Feb. 27, 2002 and entitled “Surgical Navigation Systems and Processes for Unicompartmental Knee Arthroplasty”; International Application No. US02/05783 entitled “Surgical Navigation Systems and Processes for High Tibial Osteotomy”; U.S. Ser. No. 10/364,859, filed Feb. 11, 2003 and entitled “Image Guided Fracture Reduction,” which claims priority to U.S. Ser. No. 60/355,886, filed Feb. 11, 2002 and entitled “Image Guided Fracture Reduction”; U.S. Ser. No. 60/271,818, filed Feb. 27, 2001 and entitled “Image Guided System for Arthroplasty”; and U.S. Ser. No. 10/229,372, filed Aug. 27, 2002 and entitled “Image Computer Assisted Knee Arthroplasty”, the entire contents of each of which are incorporated herein by reference as are all documents incorporated by reference therein.

These systems and processes use position and/or orientation tracking sensors such as infrared sensors acting stereoscopically or other sensors acting in conjunction with navigational references to track positions of body parts, surgery-related items such as implements, instrumentation, trial prosthetics, prosthetic components, and virtual constructs or references such as rotational axes which have been calculated and stored based on designation of bone landmarks. Sensors, such as cameras, detectors, and other similar devices, are typically mounted overhead with respect to body parts and surgery-related items to receive, sense, or otherwise detect positions and/or orientations of the body parts and surgery-related items. Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated navigational references, or based on stored position and/or orientation information. The processing functionality correlates this position and orientation information for each object with stored information, such as a computerized fluoroscopic imaged file, a wire frame data file for rendering a representation of an instrument component, trial prosthesis or actual prosthesis, or a computer generated file relating to a reference, mechanical, rotational or other axis or other virtual construct or reference. The processing functionality then displays position and orientation of these objects on a rendering functionality, such as a screen, monitor, or otherwise, in combination with image information or navigational information such as a reference, mechanical, rotational or other axis or other virtual construct or reference. Thus, these systems or processes, by sensing the position of navigational references, can display or otherwise output useful data relating to predicted or actual position and orientation of surgical instruments, body parts, surgically related items, implants, and virtual constructs for use in navigation, assessment, and otherwise performing surgery or other operations.

Some of the navigational references used in these systems may emit or reflect infrared light that is then detected by an infrared sensor. The references may be sensed actively or passively by infrared, visual, sound, magnetic, electromagnetic, x-ray or any other desired technique. An active reference emits energy, and a passive reference merely reflects energy. Some navigational references may have markers or fiducials that are traced by an infrared sensor to determine the position and orientation of the reference and thus the position and orientation of the associated instrument, item, implant component or other object to which the reference is attached.

In addition to navigational references with fixed fiducials, modular fiducials, which may be positioned independent of each other, may be used to reference points in the coordinate system. Modular fiducials may include reflective elements which may be tracked by two, sometimes more, sensors whose output may be processed in concert by associated processing functionality to geometrically calculate the position and orientation of the item to which the modular fiducial is attached. Like fixed fiducial navigational references, modular fiducials and the sensors need not be confined to the infrared spectrum-any electromagnetic, electrostatic, light, sound, radio frequency or other desired technique may be used. Similarly, modular fiducials may “actively” transmit reference information to a tracking system, as opposed to “passively” reflecting infrared or other forms of energy.

Navigational references useable with the above-identified navigation systems may be secured to any desired structure, including the above-mentioned surgical instruments and other items. The navigational references may be secured directly to the instrument or item to be referenced. However, in many instances it will not be practical or desirable to secure the navigational references to the instrument or other item. Rather, in many circumstances it will be preferred to secure the navigational references to a handle and/or a guide adapted to receive the instrument or other item. For example, drill bits and other rotating instruments cannot be tracked by securing the navigational reference directly to the rotating instrument because the reference would rotate along with the instrument. Rather, a preferred method for tracking a rotating instrument is to associate the navigational reference with the instrument or item's guide or handle.

Some or all of the computer-aided surgical navigation systems disclosed above can be used in conjunction with various surgeries to provide surgical-related information during surgery. For example, some computer-aided surgical navigation systems can be used to assist a user, such as a surgeon, in positioning, aligning, and installing an acetabular cup or component relative to a patient's pelvic bone in a hip replacement surgery. In some conventional surgical procedures for hip replacement surgery, time consuming or invasive procedures may be needed to identify a relevant reference plane for orienting an acetabular cup or component with respect to the patient's pelvis. Conventional surgical procedures performed in conjunction with or using computer-aided surgical navigational systems can require time consuming, invasive, or inaccurate procedures to reference the acetabular cup or component position relative to an anterior pelvic plane of a patient's pelvic bone.

For example, in some hip replacement procedures, a pre-operative computer tomography (CT) imaging scan may be performed prior to the hip replacement surgery. Such a scan can require extensive pre-operative planning, which incurs additional cost and time for the patient and associated medical personnel. Nevertheless, even if a CT scan is performed prior to a hip replacement surgery, there can sometimes be difficulty in the intra-operative registration of the patient to the data collected by the CT scan. In other hip replacement surgeries, an intraoperative fluoroscopy may be performed. However, there can sometimes be difficulty in obtaining useful intraoperative fluoroscopy images, and even if useful images are obtained, in some instances there may be difficulty in identifying suitable landmarks or reference points on the patient's pelvic bone or body. In other hip replacement surgeries, direct palpation of the patient's pelvic bone may require the use of a sharp probe to digitize a bony landmark on the bone. In some instances, an additional wound on the patient is created, and sometimes there may be errors in identifying certain landmarks due to the relatively blind nature in selecting a relevant landmark on the patient's bone covered by the patient's skin.

In each of the conventional procedures described above, a degree of inaccuracy in identifying the patient's pelvic plane for orienting an acetabular cup or component can be introduced, which may lead to premature failure of the acetabular cup or component, or other hip replacement component if the acetabular cup or component is not suitably aligned with the patient's pelvic bone.

### SUMMARY OF THE INVENTION

Systems and methods according to various embodiments of the invention address some or all of the above issues and combinations thereof. They do so by providing a computer-aided surgical system, methods, and associated surgical methods for providing a reference plane for mounting an acetabular cup or component during a computer-aided surgical procedure. During a computer-aided surgery, the computer-aided surgical system, methods, and associated surgical methods can improve the alignment of an acetabular cup or component with respect to a patient's pelvic bone prior to and during a computer-aided surgical procedure, such as a hip replacement. Such systems and methods are particularly useful for surgeons installing orthopedic components within a patient's body, wherein the computer-aided surgical navigation system can identify and display a relevant plane for a surgeon to reference during positioning and alignment of an acetabular cup or component with respect to a patient's pelvic bone. Essentially, a patient is positioned in a supine position on an upper surface of a platform, such as a surgical table. One or more arrays or navigational references can be mounted to the upper surface of the platform to define a reference plane. This reference plane can be used as a substitute or proxy for the anterior pelvic plane, which is associated with the patient's pelvic bone. Other arrays or navigational references can be mounted to the patient's pelvic bone to define a second reference plane. Using either or both reference planes, a surgeon using a computer-aided surgical procedure can align and mount an acteabular cup or component with respect to the patient's pelvic bone.

One aspect of systems, methods, and apparatuses according to various embodiments of the invention, focuses on computer-aided surgical navigational system with a display screen and at least one sensor. The system can include a processor capable of detecting at least one array associated with a platform upon which the patient is supported in a supine position. In addition, the processor is capable of defining a reference plane for a surgical procedure associated with an acetabular component, based at least in part on detecting the array associated with the platform using the sensor. Furthermore, the processor is capable of outputting via the screen at least one user interface adapted to use with the surgical procedure associated with an acetabular component, based at least in part on defining the reference plane.

According to another aspect of the invention, systems, methods, and apparatuses according to various embodiments of the invention include a method performed by a computer-aided surgical navigational system with a display screen and at least one sensor. The method can include detecting at least one array associated with a platform upon which the patient is supported in a supine position. The method can also include defining a reference plane for a surgical procedure associated with an acetabular component, based at least in part on detecting the array associated with the platform. Furthermore, the method can include outputting via the screen at least one user interface for use with the surgical procedure associated with an acetabular component, based at least in part on detecting the array associated with the portion of the platform.

According to another aspect of the invention, systems, methods, and apparatuses according to various embodiments of the invention include a a computer-aided surgical navigational system with a display screen and at least one sensor. The system can include a processor capable of detecting at least one array associated with a portion of a patient's pelvic bone while the patient is in a supine position. The processor is further capable of detecting at least one array associated with a platform upon which the patient is supported in a supine position. In addition, the processor is capable of defining a surgical reference plane for a surgical procedure associated with an acetabular component, based at least in part on detecting the array associated with the platform using the sensor. Furthermore, the processor is capable of outputting via the screen at least one user interface adapted to use with the surgical procedure associated with an acetabular component, based at least in part on detecting the array associated with the portion of a patient's pelvic bone using the sensor, and further based at least in part on detecting the array associated with the platform using the sensor.

According to another aspect of the invention, systems, methods, and apparatuses according to various embodiments of the invention include a method performed by a computer-aided surgical navigational system with a display screen and at least one sensor. The method can include detecting at least one array associated with a portion of a patient's pelvic bone while the patient is in a supine position. In addition, the method can include detecting at least one array associated with a platform upon which the patient is supported in a supine position. The method can also include defining a reference plane for a surgical procedure associated with an acetabular component, based at least in part on detecting the array associated with the platform using the sensor. Furthermore, the method can include outputting via the screen at least one user interface for use with the surgical procedure associated with an acetabular component, based at least in part on detecting the array associated with the portion of a patient's pelvic bone using the sensor, and further based at least in part on detecting the array associated wit the platform using the sensor.

According to yet another aspect of the invention, systems, methods, and apparatuses according to various embodiments of the invention can include a surgical method performed in conjunction with a computer-aided surgical navigational system with a display screen and at least one sensor. The surgical method can include providing a platform with an upper surface capable of supporting a patient in a supine position. In addition, the surgical method can include orienting a patient in a supine position upon the upper surface of the platform. Furthermore, the surgical method can include positioning an array with respect to the upper surface of the platform, wherein the array can be detected by the at least one sensor. Furthermore, the surgical method can include defining a reference plane for a surgical procedure associated with an acetabular component, based at least in part on detecting the position of the array associated with the position of the upper surface of the platform using the sensor.

According to yet another aspect of the invention, systems, methods, and apparatuses according to various embodiments of the invention can include a surgical method performed in conjunction with a computer-aided surgical navigational system with a display screen and at least one sensor. The surgical method can include orienting a patient in a supine position adjacent to an upper surface of a platform. In addition, the surgical method can include defining a first reference plane with respect to a portion of a patient's pelvic bone, wherein the first reference plane can be detected by at least one sensor. In addition, the surgical method can include defining a second reference plane with respect to the upper surface of the platform, wherein the second reference plane can be detected by the at least one sensor. Moreover, the surgical method can include performing a surgical procedure associated with an acetabular component, based at least in part on the first reference plane and the second reference plane.

Objects, features and advantages of various systems, methods, and apparatuses according to various embodiments of the invention include:

(1) providing the ability to obtain a reference plane for an acetabular cup or component during a computer-aided surgery;

(2) providing the ability to obtain a reference plane for aligning and mounting an acetabular cup or component with respect to a patient's pelvic bone during a computer-aided surgery;

(3) providing the ability for a user to obtain a reference for an acetabular cup or component during a computer-aided surgery; and

(4) providing the ability for a user to obtain a reference plane for aligning and mounting an acetabular cup or component with respect to a patient's pelvic bone during a computer-aided surgery.

Other aspects, features and advantages of various aspects and embodiments of systems and methods according to the invention are apparent from the other parts of this document.

### BRIEF DESCRIPTION OF THE DRAWINGS

FIG. 1 is an exemplary environment for a computer-aided surgical navigational system in accordance with an embodiment of the invention.

FIG. 2 is an example position of an array to define a reference plane with respect to an upper surface of a platform upon which a patient can be supported in a supine position in accordance with an embodiment of the invention.

FIG. 3 is an example position of an array to define a reference plane with respect to a patient in a supine position on a platform, and a second array to define a second reference plane with respect to a patient's pelvic bone in accordance with an embodiment of the invention.

FIG. 4 is an example of an acetabular component being oriented with respect to a patient's pelvic bone in accordance with an embodiment of the invention.

FIG. 5 is an example of an acetabular component oriented with respect to a patient's pelvic bone in accordance with an embodiment of the invention.

FIG. 6 is a flowchart for a method capable of being performed in conjunction with the computer-aided surgical navigational system shown in FIG. 1.

FIG. 7 is a flowchart for a method used in conjunction with the computer-aided surgical navigational system shown in FIG. 1.

FIG. 8 is a flowchart for a surgical method used in conjunction with the computer-aided surgical navigational system according to another embodiment of the invention.

FIG. 9 is a flowchart for another surgical method used in conjunction with the computer-aided surgical navigational system according to another embodiment of the invention.

### DETAILED DESCRIPTION OF SPECIFIC EMBODIMENTS

Systems and methods according to various embodiments of the invention address some or all of the above issues and combinations thereof. They do so by providing a computer-aided surgical system and methods which can improve the alignment of an acetabular cup or component with a patient's pelvic bone during a computer-aided surgical procedure, such as a hip replacement. Such systems and methods are particularly useful for surgeons installing an orthopedic component, such as a acetabular cup or component, within a patient's body, wherein the computer-aided surgical navigation system can identify and display a relevant plane for a surgeon to reference during alignment and mounting of an acetabular cup or component with respect to a patient's pelvic bone.

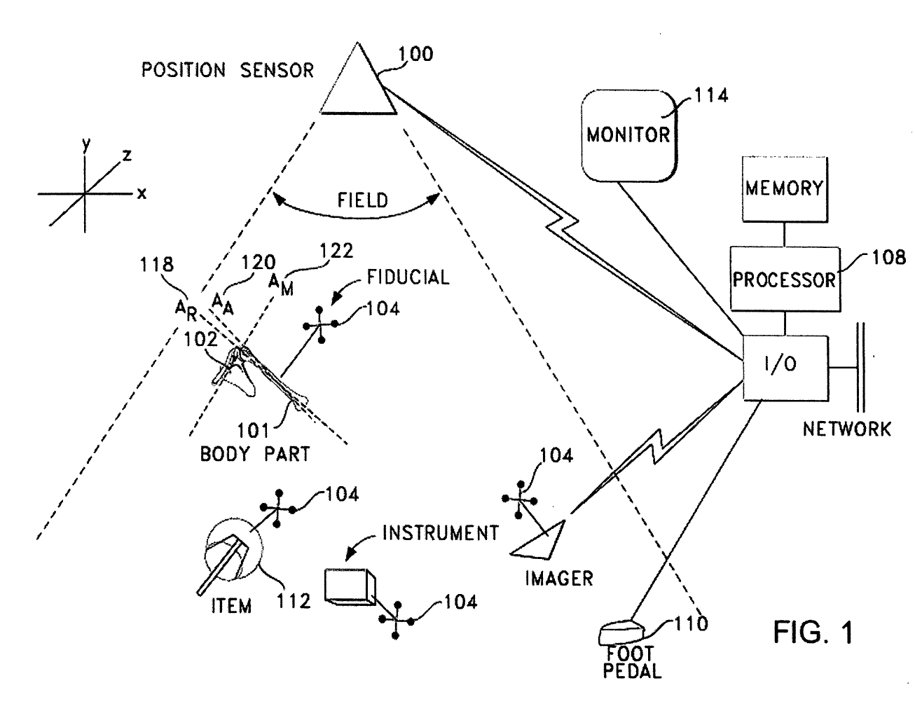

FIG. 1 is a schematic view showing an environment for using a computer-aided surgical navigation system according to some embodiments of the present invention, such as a surgery on a hip, in this case a hip arthroplasty. Systems and processes according to some embodiments of the invention can track various body parts such as a pelvic bone 101 and femur 102 to which navigational sensors 100 may be implanted, attached or associated physically, virtually or otherwise.

Navigational sensors 100 may be used to determine and track the position of body parts, axes of body parts, implements, instrumentation, trial components and prosthetic components. Navigational sensors 100 may use infrared, electromagnetic, electrostatic, light sound, radio frequency or other desired techniques.

The navigational sensor 100 may be used to sense the position and orientation of navigational references 104 and therefore items with which they are associated. A navigational reference 104 can include fiducial markers, such as marker elements, capable of being sensed by a navigational sensor in a computer-aided surgical navigation system. The navigational sensor 100 may sense active or passive signals from the navigational references 104. The signals may be electrical, magnetic, electromagnetic, sound, physical, radio frequency, optical or visual, or other active or passive technique. For example in one embodiment, the navigational sensor 100 can visually detect the presence of a passive-type navigational reference. In an example of another embodiment, the navigational sensor 100 can receive an active signal provided by an active-type navigational reference. The surgical navigation system can store, process and/or output data relating to position and orientation of navigational references 104 and thus, items or body parts, such as 101 and 102 to which they are attached or associated.

In the embodiment shown in FIG. 1, computing functionality 108 such as one or more computer programs can include processing functionality, memory functionality, input/output functionality whether on a standalone or distributed basis, via any desired standard, architecture, interface and/or network topology. In one embodiment, computing functionality 108 can be connected to a display screen or monitor 114 on which graphics, data, and other user interfaces may be presented to a surgeon during surgery. The display screen or monitor 114 preferably has a tactile user interface so that the surgeon may point and click on the display screen or monitor 114 for tactile screen input in addition to or instead of, if desired, keyboard and mouse conventional interfaces.

Additionally, a foot pedal 110 or other convenient interface may be coupled to computing functionality 108 as can any other wireless or wireline interface to allow the surgeon, nurse or other user to control or direct functionality 108 in order to, among other things, capture position/orientation information when certain components are oriented or aligned properly. Items 112 such as trial components, instrumentation components may be tracked in position and orientation relative to body parts 101 and 102 using one or more navigational references 104.

Computing functionality 108 can, but need not, process, store and output on the display screen or monitor 114 various forms of data that correspond in whole or part to body parts 101 and 202 and other components for item 112. For example, body parts 101 and 102 can be shown in cross-section or at least various internal aspects of them such as bone canals and surface structure can be shown using fluoroscopic images. These images can be obtained using an imager 113, such as a C-arm attached to a navigational reference 104. The body parts, for example, pelvic bone 101 and femur 102, can also have navigational references 104 attached. When fluoroscopy images are obtained using the C-arm with a navigational reference 104, a navigational sensor 100 “sees” and tracks the position of the fluoroscopy head as well as the positions and orientations of the pelvic bone 101 and femur 102. The computer stores the fluoroscopic images with this position/orientation information, thus correlating position and orientation of the fluoroscopic image relative to the relevant body part or parts. Thus, when the pelvic bone 101 and corresponding navigational reference 104 move, the computer automatically and correspondingly senses the new position of pelvic bone 101 in space and can correspondingly move implements, instruments, references, trials and/or implants on the monitor 114 relative to the image of pelvic bone 101. Similarly, the image of the body part can be moved, both the body part and such items may be moved, or the on-screen image otherwise presented to suit the preferences of the surgeon or others and carry out the imaging that is desired. Similarly, when an item 112, such as a stylus, cutting block, reamer, drill, saw, extramedullary rod, intramedullar rod, or any other type of item or instrument, that is being tracked moves, its image moves on monitor 114 so that the monitor 114 shows the item 112 in proper position and orientation on monitor 114 relative to the pelvic bone 101. The item 112 can thus appear on the monitor 114 in proper or improper alignment with respect to the mechanical axis and other features of the pelvic bone 101, as if the surgeon were able to see into the body in order to navigate and position item 112 properly.

The computing functionality 108 can also store data relating to configuration, size and other properties of items 112 such as joint replacement prostheses, implements, instrumentation, trial components, implant components and other items used in surgery. When those are introduced into the field of position/orientation sensor 100, computing functionality 108 can generate and display overlain or in combination with the fluoroscopic images of the body parts 101 and 102, computer generated images of joint replacement prostheses, implements, instrumentation components, trial components, implant components and other items 112 for navigation, positioning, assessment and other uses.

Instead of or in combination with fluoroscopic, MRI or other actual images of body parts, computing functionality 108 may store and output navigational or virtual construct data based on the sensed position and orientation of items in the surgical field, such as surgical instruments or position and orientation of body parts. For example, display screen or monitor 114 can output a resection plane, anatomical axis, mechanical axis, anterior/posterior reference plane, medial/lateral reference plane, rotational axis or any other navigational reference or information that may be useful or desired to conduct surgery. In the case of the reference plane, for example, display screen or monitor 114 can output a resection plane that corresponds to the resection plane defined by a cutting guide whose position and orientation is being tracked by navigational sensors 100. In other embodiments, display screen or monitor 114 can output a cutting track based on the sensed position and orientation of a reamer. Other virtual constructs can also be output on the display screen or monitor 114, and can be displayed with or without the relevant surgical instrument, based on the sensed position and orientation of any surgical instrument or other item in the surgical field to assist the surgeon or other user to plan some or all of the stages of the surgical procedure.

In some embodiments of the present invention, computing functionality 108 can output on the display screen or monitor 114 the projected position and orientation of an implant component or components based on the sensed position and orientation of one or more surgical instruments associated with one or more navigational references 104. For example, the system may track the position and orientation of a cutting block as it is navigated with respect to a portion of a body part that will be resected. Computing functionality 108 may calculate and output on the display screen or monitor 114 the projected placement of the implant in the body part based on the sensed position and orientation of the cutting block, in combination with, for example, the mechanical axis of the tibia and/or the knee, together with axes showing the anterior/posterior and medial/lateral planes. No fluoroscopic, MRI or other actual image of the body part is displayed in some embodiments, since some hold that such imaging is unnecessary and counterproductive in the context of computer aided surgery if relevant axis and/or other navigational information is displayed. Additionally, some systems use “morphed” images that change shape to fit data points or they use generic graphics or line art images with the data points displayed in a relatively accurate position or not displayed at all. If the surgeon or other user is dissatisfied with the projected placement of the implant, the surgeon may then reposition the cutting block to evaluate the effect on projected implant position and orientation.

The computer functionality 108 shown in FIG. 1 can also recognize certain surgical instruments or other objects by the navigational references 104 associated with the particular instruments. In one embodiment, this can be accomplished by storing information associated with a particular surgical instrument in memory of the computer functionality 108, and associating a discrete or unique navigational reference, such as 104, with the surgical instrument. The navigational reference, such as 104, can have a characteristic that can uniquely identify one navigational reference from another. A characteristic can include, but is not limited to, a shape, a size, a type, or a signal. Such characteristics can be stored by the computer functionality 108, and when the computer functionality 108 detects a particular previously stored characteristic for a navigational reference, such as 104, the computer functionality 108 can identify the surgical instrument associated with the navigational reference.

Additionally, computer functionality 108 can track any point in the navigational sensor 100 field such as by using a designator or a probe 116. The probe also can contain or be attached to a navigational reference 104. The surgeon, nurse, or other user touches the tip of probe 116 to a point such as a landmark on bone structure and actuates the foot pedal 110 or otherwise instructs the computer 108 to note the landmark position. The navigational sensor 100 “sees” the position and orientation of navigational reference 104 “knows” where the tip of probe 116 is relative to that navigational reference 104 and thus calculates and stores, and can display on the display screen or monitor 114 whenever desired and in whatever form or fashion or color, the point or other position designated by probe 116 when the foot pedal 110 is hit or other command is given. Thus, probe 116 can be used to designate landmarks on bone structure in order to allow the computer 108 to store and track, relative to movement of the navigational reference 104, virtual or logical information such as retroversion axis 118, anatomical axis 120 and mechanical axis 122 of femur 102, pelvic bone 101 and other body parts in addition to any other virtual or actual construct or reference.

In one embodiment, a tip of the probe 116 can be used to touch or otherwise contact at least three points on an upper surface of a surgical table or platform. In this manner, based at least in part on the three points on the upper surface of the surgical table or platform, the probe 116 and computing functionality 108 can identify or otherwise define a reference plane associated with the upper surface of the surgical table or platform.

In one embodiment, a probe 116 can include a multi-point head, such as a tripod-shaped head, with a respective contact adjacent to the ends of or points of the head. For example, a tripod-shaped head can have three contacts adjacent to the respective ends of the head. In any instance, a multi-point head configuration can be manipulated by a user, such as a surgeon, and placed on or otherwise contacted with an upper surface of a surgical table or platform, such that the contacts make simultaneous contact with the upper surface of the surgical table or platform. In this manner, the probe 116 and computing functionality 108 can identify or otherwise define a reference plane associated with the upper surface of the surgical table or platform.

Systems and processes according to some embodiments of the present invention can communicate with suitable computer-aided surgical systems and processes such as the BrainLAB VectorVision system, the OrthoSoft Navitrack System, the Stryker Navigation system, the FluoroNav system provided by Medtronic Surgical Navigation Technologies, Inc. and software provided by Medtronic Sofamor Danek Technologies. Such systems or aspects of them are disclosed in U.S. Pat. Nos. 5,383,454; 5,871,445; 6,146,390; 6,165,81; 6,235,038 and 6,236,875, and related (under 35 U.S.C. Section 119 and/or 120) patents, which are all incorporated herein by this reference. Any other desired systems and processes can be used as mentioned above for imaging, storage of data, tracking of body parts and items and for other purposes.

These systems may require the use of reference frame type fiducials which have three or four, and in some cases five elements, tracked by sensors for position/orientation of the fiducials and thus of the body part, implement, instrumentation, trial component, implant component, or other device or structure being tracked. Such systems can also use at least one probe which the surgeon can use to select, designate, register, or otherwise make known to the system a point or points on the anatomy or other locations by placing the probe as appropriate and signaling or commanding the computer to note the location of, for instance, the tip of the probe. These systems also may, but are not required to, track position and orientation of a C-arm used to obtain fluoroscopic images of body parts to which fiducials have been attached for capturing and storage of fluoroscopic images keyed to position/orientation information as tracked by the sensors. Thus, the display screen or monitor can render fluoroscopic images of bones in combination with computer generated images of virtual constructs and references together with implements, instrumentation components, trial components, implant components and other items used in connection with surgery for navigation, resection of bone, assessment and other purposes.

In one embodiment, a series of arrays or navigational references, such as 104, can be mounted or otherwise positioned with respect to an upper surface of a surgical table or platform. In this manner, the series of arrays or navigational references can identify or otherwise define a reference plane associated with the upper surface of the surgical table or platform. The reference plane can be used as a proxy or substitute for a patient's anterior pelvic plane associated with the patient's pelvic bone during a computer-aided surgical procedure, such as mounting an acetabular cup or component with respect to a patient's pelvic bone, such as 101. In another embodiment, a series of arrays or navigational references, such as 104, can be mounted or otherwise positioned with respect to a patient's pelvic bone, such as 101. In this manner, the series of arrays or navigational references can identify or otherwise define a reference plane associated with the patient's pelvic bone. Using either or both of the reference planes associated with a surgical table or platform and with a patient's pelvic bone, the computing functionality 108 can display graphics, text, quantitative measurements, commands, or other surgical information with respect to either or both reference planes via the monitor 114 as a patient is moved or rotated on the surgical table or platform.

Other arrangements or configurations of arrays or navigational references can be used to identify or otherwise define any number of suitable reference planes for use with a computer-aided surgical procedure to align and mount an acetabular cup or component with respect to a patient's pelvic bone in accordance with embodiments of the invention.

In yet another embodiment, the computer functionality 108 can provide data to permit navigation of a surgical instrument, orthopedic device, or item, such as 112, by a user performing a surgical procedure relative to a series of arrays or navigational references, such as 104, mounted or otherwise positioned with respect to an upper surface of a surgical table or platform. Data can include, but is not limited to, text, graphics, a command, a screen display, or other information. For example, when a user, such as a surgeon, manipulates an item 112, the computer functionality 108 can receive position information associated with the item 112. Information associated with the arrays or navigational references, such as 104, mounted or otherwise positioned with respect to an upper surface of a surgical table or platform can also be received by the computer functionality 108. The computer functionality 108 can process the position information associated with the item 112 and the arrays or navigational references 104, and can coordinate the position information with previously stored data, or with software programs or routines, to provide instructions or other direction to the user to navigate the item 112 relative to the upper surface of a surgical table or platform associated in a surgical procedure.

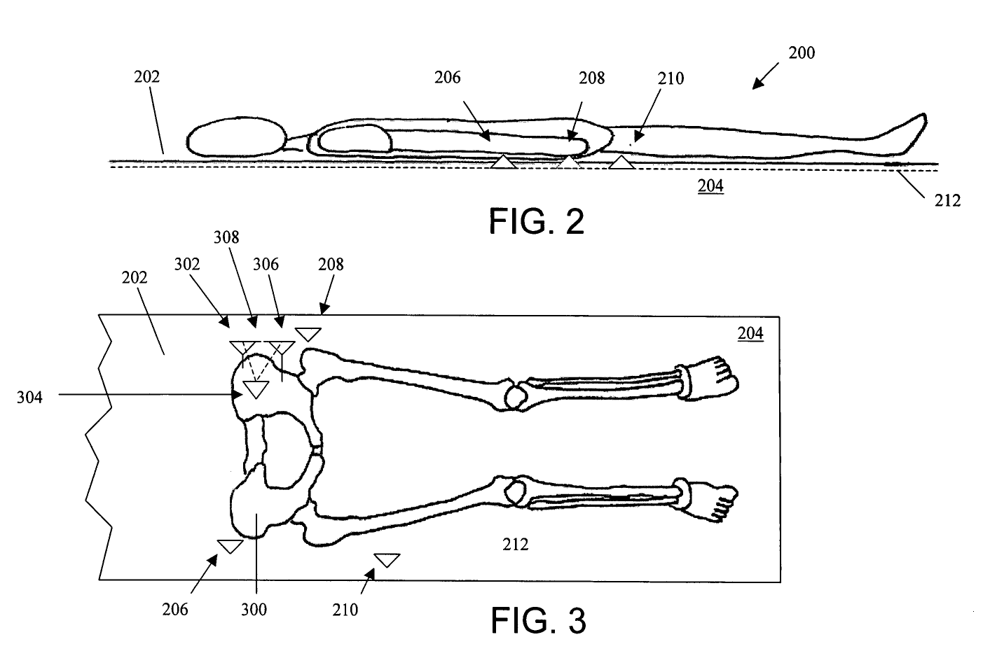

FIGS. 2-3 illustrate exemplary positions of arrays or navigational references positioned with respect to a patient's pelvic bone and a platform in accordance with embodiments of the invention. The positions of arrays or navigational references shown in FIGS. 2-3 can be used in conjunction with the computer-aided surgical navigational system shown in FIG. 1. Furthermore, either or both of the positions of arrays or navigational references shown in FIGS. 2-3 can be used in a surgical procedure, or in steps of a surgical procedure, such as aligning and mounting an acetabular cup or component with respect to a pelvic bone in a hip replacement. As explained in greater detail below, either or both of the series of arrays or navigational references shown in FIGS. 2-3 can be used to define suitable reference planes for use in a computer-aided surgical procedure associated with an acetabular cup or component. Other positions of arrays or navigational references positioned with respect to a patient's pelvic bone and/or a platform can exist in accordance with other embodiments of the invention. Furthermore, any number, shape, or configuration of arrays or navigational references can be used to define suitable reference planes for use in aligning an acetabular cup or component with respect to a patient's pelvic bone in a surgical procedure in accordance with other embodiments of the invention.

FIG. 2 illustrates a side view of a patient 200 in a supine position on an upper surface 202 of a platform 204. In the embodiment shown in FIG. 2, the platform is a surgical table. When the patient 200 is oriented in this position, the patient's pelvic bone (shown as 300 in FIG. 3) becomes oriented with respect to the upper surface 202 of a platform 204. In the embodiment shown in FIG. 2, a series of arrays 206, 208, 210 or navigational references can be mounted to the upper surface 202 of the platform 204. A sensor or position sensor, shown as 100 in FIG. 1, can identify or otherwise determine the position of the arrays 206, 208, 210 or navigational references, and can define a reference plane, such as a table plane 212, associated with the upper surface 202 of the platform 204. In one embodiment, the patient 200 can be secured to the upper surface 202 of the platform 204 with a series of straps, restraints, or other similar devices. In this manner, the reference plane defined by the arrays 206, 208, 210 can be used as a proxy or substitute for an anterior pelvic plane associated with the patient 200. Using a computer-aided surgical navigation system, such as shown in FIG. 1, a user such as a surgeon can use the reference plane in a surgical procedure, such as mounting an acetabular cup or component with respect to a patient's pelvic bone. In other embodiments, the table plane 212 may be different than illustrated, or may be another angle other than substantially horizontal, depending on the configuration of the upper surface 202 of the platform 204, or the mounting of the arrays or navigational references with respect to the upper surface 202 of the platform 204.

FIG. 3 illustrates an overhead skeletal view of a lower portion of the patient of FIG. 2 in a supine position. In this view, the patient's pelvic bone 300 can be referenced with a series of arrays 302, 304, 306 or navigational references mounted to respective portions of the pelvic bone 300. A sensor or position sensor, shown as 100 in FIG. 1, can identify or otherwise determine the position of the arrays 302, 304, 306 or navigational references, and can define a reference plane, such as a pelvic plane 308, associated with the patient's pelvic bone 300. In this manner, the reference plane defined by the arrays 302, 304, 306 can be used in conjunction with another reference plane, such as a table plane 212 described above in FIG. 2. Using a computer-aided surgical navigation system, such as shown in FIG. 1, a user such as a surgeon can use the reference planes in a surgical procedure, such as mounting an acetabular cup or component with respect to a patient's pelvic bone. In other embodiments, the pelvic plane 308 may be different than illustrated depending on the configuration of the patient's pelvic bone, or the mounting of the arrays or navigational references with respect to the patient's pelvic bone.

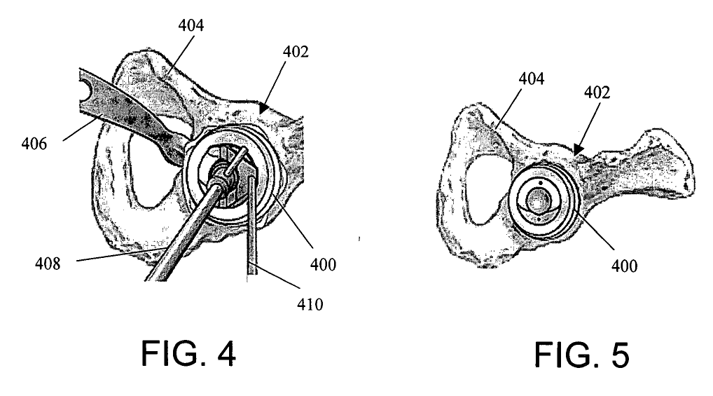

In the embodiment shown in FIGS. 2-3, the patient 200 is oriented in a supine position on the upper surface 202 of the platform 204 to establish a more clinically relevant plane, such as the table plane 212, for orienting an acetabular component, such as 400 in FIGS. 4-5, with respect to a patient's pelvic bone 300. Since the table plane 212 or other reference plane associated with the upper surface 202 of the platform 204 is not influenced or otherwise affected by the pelvic tilt of the patient's pelvic bone, or any rotation of the pelvic bone caused by spinal or pelvic deformity, or joint contracture, the table plane 212 or other reference plane associated with the upper surface 202 of the platform 204 can be better suited for assisting a surgeon in a computer-aided surgical procedure, such as installing, mounting, or orienting an acetabular component with respect to a patient's pelvic bone. Furthermore, the table plane 212 or other reference plane associated with the upper surface 202 of the platform 204 can be identified with relatively greater accuracy than other reference planes not associated with the upper surface 202 of the platform 204, such as an anterior pelvic plane.

FIGS. 4 and 5 illustrate a surgical procedure to align and mount an acetabular component with respect to a pelvic bone in a hip replacement surgery. FIG. 4 shows an acetabular component 400 being aligned and mounted with respect to an acetabulum portion 402 of a pelvic bone 404. As shown in FIGS. 2 and 3, a surgeon or other medical personnel can utilize a computer-aided surgical navigation system shown in FIG. 1 to determine one or more reference planes, such as a reference plane associated with a surgical table. Based in part on at least the reference plane associated with the surgical table, the surgeon can introduce an acetabular component 400 into the acetabulum portion 402 of a pelvic bone 404. With the assistance of one or more surgical instruments, such as a teardrop retractor 406, a cup introducer 408, and/or a cup positioner 410, the surgeon can align, mount, and install the acetabular component 400 with respect to the acetabulum portion 402 of the pelvic bone 404 based in part on at least the reference plane associated with the surgical table. In some instances, one or more surgical instruments can include respective arrays or navigational references to facilitate monitoring and alignment of the instruments with respect to any predefined reference planes, such as the reference plane associated with the surgical table. Other surgical instruments, tools, or surgical-related items can be used to align, mount, and install an acetabular component with respect to a pelvic bone in accordance with embodiments of the invention. An example of an acetabular component 400 mounted with respect to an acetabulum portion 402 of a patient's pelvic bone 404 is shown in FIG. 5.

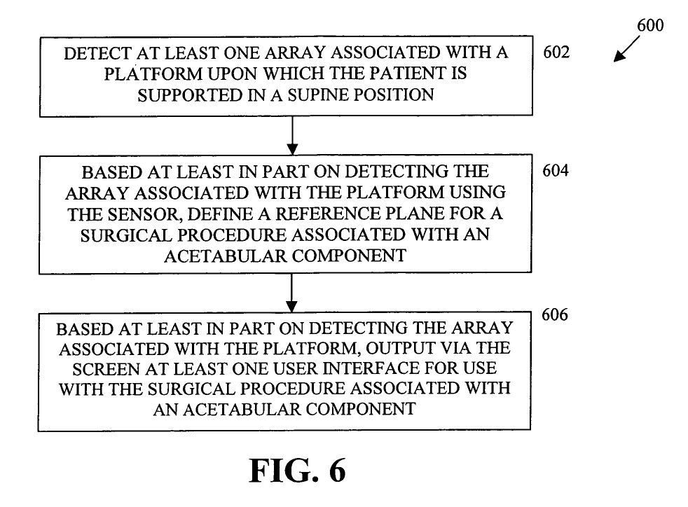

FIG. 6 illustrates a method performed by the computer-aided surgical navigational system shown in FIG. 1. The system, as described in FIG. 1, includes a display screen or monitor 114 and at least one sensor or position sensor 100. Other system embodiments can be used with the method 600 in accordance with other embodiments of the invention. Other method embodiments can have fewer or greater numbers of elements in accordance with other embodiments of the invention. The method 600 begins at block 602.

In block 602, at least one array associated with a platform upon which the patient is supported in a supine position is detected. In the embodiment shown in FIG. 6, a processor such as 108 in FIG. 1, can store information associated with one or more arrays or navigational references, such as a characteristic of a navigational reference, for instance 104 in FIG. 1. Each respective array or navigational reference can then be associated with a respective point or position adjacent to an upper surface of a platform, such as a surgical table. This association information can be stored by the processor 108. A sensor or position sensor, such as 100 in FIG. 1, can detect the position of any number of arrays or navigational references, such as 104, associated with the upper surface of the platform, such as a surgical table.

Block 602 is followed by block 604, in which based at least in part on detecting the array associated with the platform using the sensor, a reference plane for a surgical procedure associated with an acetabular component is defined. In the embodiment shown in FIG. 6, the processor such as 108 can identify or otherwise determine a reference plane using the positions of some or all of the arrays or navigational references, such as 104, positioned with respect to the platform. A suitable reference plane is a plane substantially parallel with the upper surface of the platform upon which the patient is in a supine position.

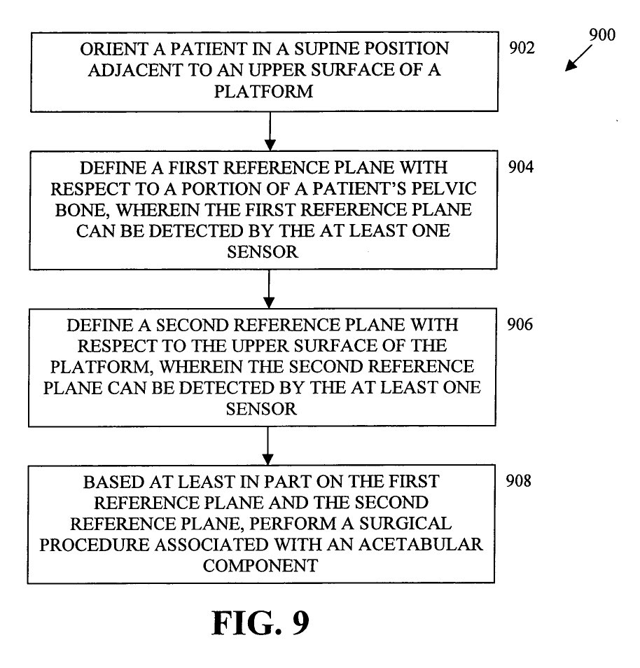

Block 604 is followed by block 606, in which based at least in part on detecting the array associated with the platform, at least one user interface for use with the surgical procedure associated with an acetabular component is output via the screen. In the embodiment shown in FIG. 6, the processor such as 108 can identify or otherwise determine a reference plane using the positions of some or all of the arrays or navigational references, such as 104, positioned with respect to the platform. A suitable reference plane is a plane substantially parallel with the upper surface of the platform upon which the patient is in a supine position. In the embodiment shown in FIG. 9, the processor such as 108 can identify or otherwise determine another reference plane using the positions of some or all of the arrays or navigational references, such as 104, positioned with respect to the patient's pelvic bone. Using at least the reference plane associated with the platform, a user such as a surgeon can perform a surgical procedure such as mounting an acetabular cup or component in a hip replacement surgery. Using a display screen or monitor, such as 114 shown in FIG. 1, associated with the computer-aided surgical navigational system, the user can view some or all of the arrays, navigational references, and reference planes in conjunction with text, graphics, measurements, or other information associated with a surgical procedure.

The method 600 ends at block 608.

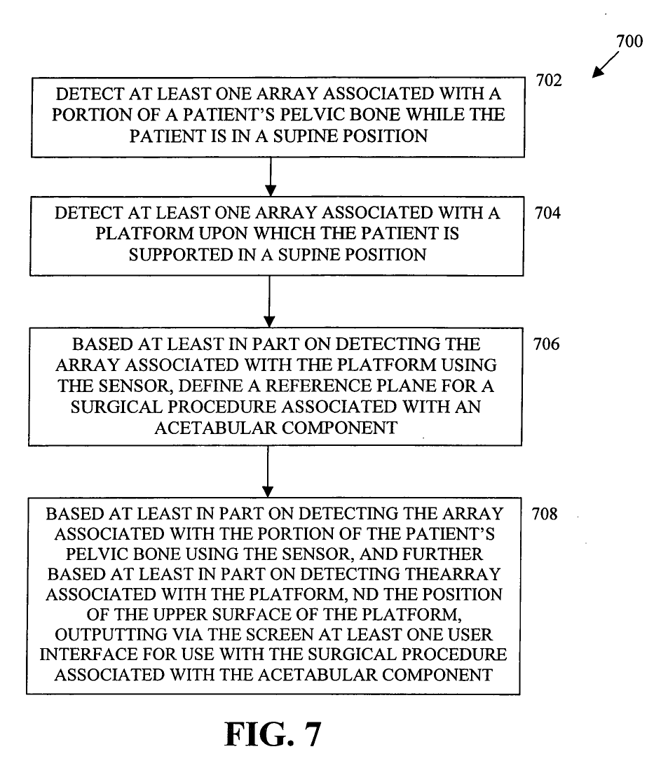

FIG. 7 illustrates a method performed by the computer-aided surgical navigational system shown in FIG. 1. The system, as described in FIG. 1, includes a display screen or monitor 114 and at least one sensor or position sensor 100. Other system embodiments can be used with the method 700 in accordance with other embodiments of the invention. Other method embodiments can have fewer or greater numbers of elements in accordance with other embodiments of the invention. The method 700 begins at block 702.

In block 702, at least one array associated with a portion of a patient's pelvic bone is detected while the patient is in a supine position. In the embodiment shown in FIG. 7, a processor such as 108 in FIG. 1, can store information associated with one or more arrays or navigational references, such as a characteristic of a navigational reference, for instance 104 in FIG. 1. Each respective array or navigational reference can then be associated with a respective point or position adjacent to a patient's pelvic bone. This association information can be stored by the processor 108. A sensor or position sensor, such as 100 in FIG. 1, can detect the position of any number of arrays or navigational references, such as 104, associated with the patient's pelvic bone.

Block 702 is followed by block 704, in which at least one array associated with a platform upon which the patient is supported in a supine position is detected. In the embodiment shown in FIG. 7, a processor such as 108 in FIG. 1, can store information associated with one or more arrays or navigational references, such as a characteristic of a navigational reference, for instance 104 in FIG. 1. Each respective array or navigational reference can then be associated with a respective point or position adjacent to an upper surface of a platform, such as a surgical table. This association information can be stored by the processor 108. A sensor or position sensor, such as 100 in FIG. 1, can detect the position of any number of arrays or navigational references, such as 104, associated with the upper surface of the platform, such as a surgical table.

Block 704 is followed by block 706, in which based at least in part on detecting the array associated with the platform using the sensor, a reference plane is defined for a surgical procedure associated with an acetabular component. In the embodiment shown in FIG. 6, the processor such as 108 can identify or otherwise determine a reference plane using the positions of some or all of the arrays or navigational references, such as 104, positioned with respect to the platform. A suitable reference plane is a plane substantially parallel with the upper surface of the platform upon which the patient is in a supine position.

Block 706 is followed by block 708, in which based at least in part on detecting the array associated with the portion of a patient's pelvic bone using the sensor, and further based at least in part on detecting the array associated with the platform with the sensor, at least one user interface for use with the surgical procedure associated with an acetabular component is output via the screen. In the embodiment shown in FIG. 7, the processor such as 108 can identify or otherwise determine a reference plane using the positions of some or all of the arrays or navigational references, such as 104, positioned with respect to the platform. A suitable reference plane is a plane substantially parallel with the upper surface of the platform upon which the patient is in a supine position. In the embodiment shown in FIG. 7, the processor such as 108 can identify or otherwise determine another reference plane using the positions of some or all of the arrays or navigational references, such as 104, positioned with respect to the patient's pelvic bone. Using at least the reference plane associated with the platform, a user such as a surgeon can perform a surgical procedure such as mounting an acetabular cup or component in a hip replacement surgery. Using a display screen or monitor, such as 114 shown in FIG. 1, associated with the computer-aided surgical navigational system, the user can view some or all of the arrays, navigational references, and reference planes in conjunction with text, graphics, measurements, or other information associated with a surgical procedure.

The method 700 ends at block 708.

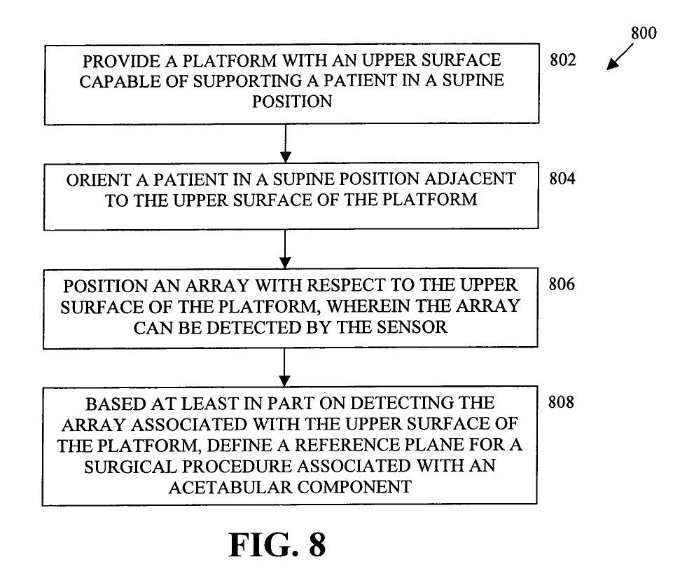

FIG. 8 illustrates a surgical method performed in conjunction with the computer-aided surgical navigational system shown in FIG. 1. The system, as described in FIG. 1, includes a display screen or monitor 114 and at least one sensor or position sensor 100. Other system embodiments can be used with the method 800 in accordance with other embodiments of the invention. Other method embodiments can have fewer or greater numbers of elements in accordance with other embodiments of the invention. The method 800 begins at block 802.

In block 802, a platform with an upper surface capable of supporting a patient in a supine position is provided. In the embodiment shown in FIG. 8, the platform can be a surgical table.

Block 802 is followed by block 804, in which a patient is oriented in a supine position upon the upper surface of the platform. In the embodiment shown in FIG. 8, a patient can be oriented, and in some instances, secured to an upper surface of the platform or surgical table.

Block 804 is followed by block 806, in which an array is positioned with respect to the upper surface of the platform, wherein the array can be detected by the sensor. In the embodiment shown in FIG. 8, a processor such as 108 in FIG. 1, can store information associated with one or more arrays or navigational references, such as a characteristic of a navigational reference, for instance 104 in FIG. 1. Each respective array or navigational reference can then be associated with a respective point or position adjacent to an upper surface of a platform, such as a surgical table. This association information can be stored by the processor 108. A sensor or position sensor, such as 100 in FIG. 1, can detect the position of any number of arrays or navigational references, such as 104, associated with the patient's pelvic bone.

Block 806 is followed by block 808, in which based at least in part on detecting the array associated with the upper surface of the platform, a reference plane for a surgical procedure associated with an acetabular component can be defined. In the embodiment shown in FIG. 6, the processor such as 108 can identify or otherwise determine a reference plane using the positions of some or all of the arrays or navigational references, such as 104, positioned with respect to the platform. A suitable reference plane is a plane substantially parallel with the upper surface of the platform upon which the patient is in a supine position. Using at least the reference plane associated with the platform, a user such as a surgeon can perform a surgical procedure such as mounting an acetabular cup or component in a hip replacement surgery. Using a display screen or monitor, such as 114 shown in FIG. 1, associated with the computer-aided surgical navigational system, the user can view some or all of the arrays, navigational references, and reference planes in conjunction with text, graphics, measurements, or other information associated with a surgical procedure.

The method 800 ends at block 808.

FIG. 9 illustrates another surgical method performed in conjunction with the computer-aided surgical navigational system shown in FIG. 1. The system, as described in FIG. 1, includes a display screen or monitor 114 and at least one sensor or position sensor 100. Other system embodiments can be used with the method 900 in accordance with other embodiments of the invention. Other method embodiments can have fewer or greater numbers of elements in accordance with other embodiments of the invention. The method 900 begins at block 902.

In block 902, a patient is oriented in a supine position adjacent to an upper surface of a platform. In the embodiment shown in FIG. 9, the platform can be a surgical table. A patient can be oriented, and in some instances, secured to an upper surface of the platform or surgical table.

Block 902 is followed by block 904, in which a first reference plane is defined with respect to a portion of a patient's pelvic bone, wherein the first reference plane can be detected by the at least one sensor. In the embodiment shown in FIG. 9, a processor such as 108 in FIG. 1, can store information associated with one or more arrays or navigational references, such as a characteristic of a navigational reference, for instance 104 in FIG. 1. Each respective array or navigational reference can then be associated with a respective point or position adjacent to a patient's pelvic bone. This association information can be stored by the processor 108. A sensor or position sensor, such as 100 in FIG. 1, can detect the position of any number of arrays or navigational references, such as 104, associated with the patient's pelvic bone.

Block 904 is followed by block 906, in which a second reference plane is defined with respect to the upper surface of the platform, wherein the second reference plane can be detected by the at least one sensor. In the embodiment shown in FIG. 9, a processor such as 108 in FIG. 1, can store information associated with one or more arrays or navigational references, such as a characteristic of a navigational reference, for instance 104 in FIG. 1. Each respective array or navigational reference can then be associated with a respective point or position adjacent to an upper surface of a platform, such as a surgical table. This association information can be stored by the processor 108. A sensor or position sensor, such as 100 in FIG. 1, can detect the position of any number of arrays or navigational references, such as 104, associated with the patient's platform.

Block 906 is followed by block 908, in which based at least in part on the first reference plane and the second reference plane, a surgical procedure associated with an acetabular component can be performed. In the embodiment shown in FIG. 6, the processor such as 108 can identify or otherwise determine a reference plane using the positions of some or all of the arrays or navigational references, such as 104, positioned with respect to the platform. A suitable reference plane is a plane substantially parallel with the upper surface of the platform upon which the patient is in a supine position. In the embodiment shown in FIG. 9, the processor such as 108 can identify or otherwise determine another reference plane using the positions of some or all of the arrays or navigational references, such as 104, positioned with respect to the patient's pelvic bone. Using at least the reference plane associated with the platform, a user such as a surgeon can perform a surgical procedure such as mounting an acetabular cup or component in a hip replacement surgery. Using a display screen or monitor, such as 114 shown in FIG. 1, associated with the computer-aided surgical navigational system, the user can view some or all of the arrays, navigational references, and reference planes in conjunction with text, graphics, measurements, or other information associated with a surgical procedure.

The method 900 ends at block 908.

While the above description contains many specifics, these specifics should not be construed as limitations on the scope of the invention, but merely as exemplifications of the disclosed embodiments. Those skilled in the art will envision many other possible variations that within the scope of the invention as defined by the claims appended hereto.

The invention claimed is:

1. A computer-aided surgical navigational system with a display screen and at least one sensor, comprising:
a processor capable of
detecting at least one array associated with a platform upon which the patient is supported in a supine position;
based at least in part on detecting the array associated with the platform using the sensor, defining a reference plane for a surgical procedure associated with an acetabular component; and
based at least in part on the defined surgical reference plane, outputting via the screen at least one user interface adapted to use with the surgical procedure associated with an acetabular component.

2. The system of claim 1, wherein the array comprises at least one of the following: a fiducial member, a sensor, an infrared sensor, or a marker.

3. The system of claim 1, wherein the acetabular component comprises at least one of the following: an acetabular cup, or an orthopedic component adapted for an acetabulum.

4. The system of claim 1, wherein the surgical procedure comprises at least one of the following: an acetabular cup installation, an acetabular component installation, or a hip arthroplasty.

5. The system of claim 1, wherein the user interface comprises at least one of the following: a display of the portion of the patient's pelvic bone relative to the platform, an instruction associated with the surgical procedure, a selection of measurements associated with the surgical procedure, or a command associated with the surgical procedure.

6. The system of claim 1, wherein detecting at least one array associated with a platform upon which the patient is supported comprises detecting contact of a probe to a portion of the platform upon which the patient is supported.

7. A method performed by a computer-aided surgical navigational system with a display screen and at least one sensor, comprising:
detecting at least one array associated with a platform upon which the patient is supported in a supine position;
based at least in part on detecting the array associated with the platform, defining a reference plane for a surgical procedure associated with an acetabular component; and
based at least in part on detecting the array associated with the platform, outputting via the screen at least one user interface for use with the surgical procedure associated with an acetabular component.

8. The method of claim 7, wherein the array comprises at least one of the following: a fiducial member, a sensor, an infrared sensor, or a marker.

9. The method of claim 7, wherein the acetabular component comprises at least one of the following: an acetabular cup, or an orthopedic component adapted for an acetabulum.

10. The method of claim 7, wherein the surgical procedure comprises at least one of the following: an acetabular cup installation, an acetabular component installation, or a hip arthroplasty.

11. The method of claim 7, wherein the user interface comprises at least one of the following: a display of the portion of the patient's pelvic bone relative to the platform, an instruction associated with the surgical procedure, a selection of measurements associated with the surgical procedure, or a command associated with the surgical procedure.

12. The method of claim 7, wherein detecting at least one array associated with a platform upon which the patient is supported in a supine position comprises detecting contact of a probe to a portion of the platform upon which the patient is supported.

13. A computer-aided surgical navigational system with a display screen and at least one sensor, comprising:
a processor capable of
detecting at least one array associated with a portion of a patient's pelvic bone while the patient is in a supine position;
detecting at least one array associated with a platform upon which the patient is supported in a supine position;
based at least in part on detecting the array associated with the platform using the sensor, defining a reference plane for a surgical procedure associated with an acetabular component; and
based at least in part on detecting the array associated with the portion of a patient's pelvic bone using the sensor, and further based at least in part on detecting the array associated with the platform using the sensor, outputting via the screen at least one user interface adapted to use with the surgical procedure associated with an acetabular component.

14. The system of claim 13, wherein each of the arrays comprise at least one of the following: a fiducial member, a sensor, an infrared sensor, or a marker.

15. The system of claim 13, wherein the acetabular component comprises at least one of the following: an acetabular cup, or an orthopedic component adapted for an acetabuium.

16. The system of claim 13, wherein the surgical procedure comprises at least one of the following: an acetabular cup installation, an acetabular component installation, or a hip arthroplasty.

17. The system of claim 13, wherein the user interface comprises at least one of the following: a display of the portion of the patient's pelvic bone relative to the platform, an instruction associated with the surgical procedure, a selection of measurements associated with the surgical procedure, or a command associated with the surgical procedure.

18. The system of claim 13, wherein detecting at least one array associated with a platform upon which the patient is supported in a supine position comprises detecting contact of a probe to a portion of the platform upon which the patient is supported.

19. A method performed by a computer-aided surgical navigational system with a display screen and at least one sensor, comprising:
detecting at least one array associated with a portion of a patient's pelvic bone while the patient is in a supine position;
detecting at least one array associated with a platform upon which the patient is supported in a supine position;
based at least in part on detecting the array associated with the platform using the sensor, defining a reference plane for a surgical procedure associated with an acetabular component; and
based at least in part on detecting the array associated with the portion of a patient's pelvic bone using the sensor, and further based at least in part on detecting the array associated with the platform using the sensor, outputting via the screen at least one user interface for use with the surgical procedure associated with an acetabular component.

20. The method of claim 19, wherein each of the arrays comprise at least one of the following: a fiducial member, a sensor, an infrared sensor, or a marker.

21. The method of claim 19, wherein the acetabular component comprises at least one of the following: an acetabular cup, or an orthopedic component adapted for an acetabulum.

22. The method of claim 19, wherein the surgical procedure comprises at least one of the following: an acetabular cup installation, an acetabular component installation, or a hip arthroplasty.

23. The method of claim 19, wherein the user interface comprises at least one of the following: a display of the portion of the patient's pelvic bone relative to the platform, an instruction associated with the surgical procedure, a selection of measurements associated with the surgical procedure, or a command associated with the surgical procedure.

24. The method of claim 19, wherein detecting at least one array associated with a platform upon which the patient is supported in a supine position comprises detecting contact of a probe to a portion of the platform upon which the patient is supported.

25. A surgical method performed in conjunction with a computer-aided surgical navigational system with a display screen and a sensor, comprising:
providing a platform with an upper surface capable of supporting a patient in a supine position;
orienting a patient in supine position adjacent to the upper surface of the platform;
positioning an array with respect to the upper surface of the platform, wherein the array can be detected by the sensor; and
based at least in part on detecting the array associated with the upper surface of the platform using the sensor, defining a reference plane for a surgical procedure associated with an acetabular component.

26. The surgical method of claim 25, wherein detecting the array associated with the platform upon which the patient is supported in a supine position using the sensor comprises contacting a probe adjacent to the upper surface of the platform.

27. The surgical method of claim 25, wherein the array comprises at least one of the following: a fiducial member, a sensor, an infrared sensor, or a marker.

28. The surgical method of claim 25, wherein orienting a patient in supine position upon the upper surface of the platform comprises securing the position of the patient relative to the upper surface of the platform.

29. The surgical method of claim 25, wherein the acetabular component comprises at least one of the following: an acetabular cup, or an orthopedic component adapted for an acetabulum.

30. The surgical method of claim 25, wherein the surgical procedure comprises at least one of the following: an acetabular cup installation, an acetabular component installation, or a hip arthroplasty.

31. The surgical method of claim 25, wherein defining a reference plane for a surgical procedure associated with an acetabular component comprises receiving via the screen at least one user interface adapted to use with the surgical procedure associated with the acetabular component.

32. The surgical method of claim 25, wherein the reference plane approximates an anterior pelvic plane associated with the patient's pelvic bone.

33. The surgical method of claim 26, wherein the user interface comprises at least one of the following: a display of the portion of the patient's pelvic bone relative to the platform, an instruction associated with the surgical procedure, a selection of measurements associated with the surgical procedure, or a command associated with the surgical procedure.

34. A surgical method performed in conjunction with a computer-aided surgical navigational system with a display screen and at least one sensor, comprising:
orienting a patient in a supine position adjacent to an upper surface of a platform;
defining a first reference plane with respect to a portion of a patient's pelvic bone, wherein the first reference plane can be detected by the at least one sensor;
defining a second reference plane with respect to the upper surface of the platform, wherein the second reference plane can be detected by the at least one sensor; and
based at least in part on the first reference plane and the second reference plane, performing a surgical procedure associated with an acetabular component.

35. The surgical method of claim 34, wherein defining a second reference plane with respect to the upper surface of the platform comprises contacting a probe adjacent to the upper surface of the platform.

36. The surgical method of claim 34, wherein the array comprises at least one of the following: a fiducial member, a sensor, an infrared sensor, or a marker.

37. The surgical method of claim 34, wherein orienting a patient in supine position adjacent to an upper surface of a platform comprises securing the position of the patient relative to the upper surface of the platform.

38. The surgical method of claim 34, wherein the acetabular component comprises at least one of the following: an acetabular cup, or an orthopedic component adapted for an acetabulum.

39. The surgical method of claim 34, wherein the surgical procedure comprises at least one of the following: an acetabular cup installation, an acetabular component installation, or a hip arthroplasty.

40. The surgical method of claim 34, wherein performing a surgical procedure associated with an acetabular component comprises receiving via the screen at least one user interface adapted to use with the surgical procedure associated with the acetabular component.

41. The surgical method of claim 34, wherein the second reference plane approximates an anterior pelvic plane associated with the patient's pelvic bone.

42. The surgical method of claim 35, wherein the user interface comprises at least one of the following: a display of the portion of the patient's pelvic bone relative to the platform, an instruction associated with the surgical procedure, a selection of measurements associated with the surgical procedure, or a command associated with the surgical procedure.
